(19) United States
(12) Patent Application Publication
(10) Pub. No.: US 2022/0252622 A1
(43) Pub. Date: Aug. 11, 2022

(54) SINGLE TUBE PREPARATION COMPRISING A PANEL OF DIFFERENTLY LABELED CELLS FOR SEROLOGY

(71) Applicant: SANQUIN REAGENTS B.V., Amsterdam (NL)

(72) Inventors: Eric Marinus Maria VAN DER DONK, Amsterdam (NL); Emile VAN DEN AKKER, Amsterdam (NL); Patrick BURGER, Amsterdam (NL)

(73) Assignee: SANQUIN REAGENTS B.V., Amsterdam (NL)

(21) Appl. No.: 17/617,343

(22) PCT Filed: Jun. 20, 2019
(86) PCT No.: PCT/EP2019/066396

§ 371 Date: Dec. 8, 2021

(51) Int. Cl. G01N 33/80; G01N 33/533; G01N 33/68

(52) U.S. Cl. CPC G01N 33/80; G01N 2015/0065; G01N 33/6854; G01N 33/533

(57) ABSTRACT

Single tube preparations include a panel of differently labeled serologically relevant blood cells for blood serology, and methods for preparing the present single tube preparations. Use of the present single tube preparations for blood serology and to methods for serologically characterizing an individual in need of a blood transfusion, a blood donor or for preventive serologically characterizing an individual, such as a pregnant woman. The single tube preparations and methods are especially suited for automation and high-through put blood serology.

The present invention relates to single tube preparations, i.e. a single container, comprising a panel of differently labeled serologically relevant blood cells for blood serology and to methods for preparing the present single tube preparations. Further, the present invention relates to the use of the present single tube preparations for blood serology and especially to methods for serologically characterizing an individual in need of a blood transfusion, a blood donor or for preventive serologically characterizing an individual, such as a pregnant woman. The present invention is especially suited for automation and/or high-through put blood serology.

In a clinical setting, a major objective of blood group serology, and, especially, blood group assays and antibody detection, is to obtain compatible erythrocyte or thrombocyte preparations from a donor for blood transfusion of a recipient. For this, assays are routinely performed such as blood group typing, antibody screening and identification, and (blood) compatibility assays by performing cross matches.

In general, the majority of these assays are based on the principle of agglutination, e.g. the formation of agglutinates of erythrocytes, carrying antibodies directed against blood group antigens present on the erythrocytes.

In a mammal, and particularly a human mammal, specific antigenic determinants on the cell membrane of erythrocytes are characterizing the so-called blood groups of said mammal. The information for expressing blood group antigens is generally genetically determined at the genomic level.

A well known, and generally used, blood group system is the so-called A, B, AB, and O system (ABO-system), discovered in 1900 by Karl Landsteiner. Different blood groups in this system are designated A, B, AB, and O.

In addition to the ABO-system, more than 400 red blood cell blood groups are known, the majority of these being clustered in blood group systems with diverging clinical significance. Specific antibodies to these other blood group systems can be formed after immunization with the corresponding blood group antigen, for example during blood transfusion or pregnancy, and may cause problems during a blood transfusion or pregnancy thereafter.

Presently, for transfusion practices, the ABO-system is the most important blood group system. This because every individual, characterized by a specific blood group, has antibodies in his serum against the blood group(s) that are not present. For example, an individual characterized as blood group A, will have anti-B antibodies in his serum, and vice versa.

These antibodies are so-called “naturally occurring” antibodies and are in general strong IgM type antibodies. The IgM antibodies are capable of causing a direct, i.e., without a bridging reagent, such as an anti-IgM antibody, complex formation or agglutination of, for example, erythrocytes exposing a blood group antigen against which the antibody is directed.

Blood transfusion reactions in an individual (recipient) induced by allo-antibodies, raised against foreign erythrocytes or red blood cells are called hemolytic transfusion reactions. This because they are generally accompanied by a strongly accelerated, and often deadly, breakdown of erythrocytes. Therefore, it of major importance to prevent hemolytic transfusion reactions by careful serological examination before a blood transfusion is performed. For this, several types of tests are generally carried out.

In general, both donor and recipient are typed for the ABO-blood group system and Rhesus D antigen. These must be identical, or compatible, for both the donor and recipient. The ABO-blood group found on the erythrocytes can, for example, be confirmed by performing a so-called reverse ABO typing test on the antibodies present in the serum.

Next, a screening of the serum of the recipient is performed for the presence of red blood cell antibodies directed against all other blood groups beside the ABO-system. If an antibody is found, it must be identified in order to select donor blood which is negative for the corresponding blood group antigen.

Finally, a cross-match between donor red blood cells and recipient serum can be performed to find out whether the donor and the recipient are indeed compatible.

In general, blood group antibodies are immunoglobulins of the IgG or IgM type. The antigen-antibody reaction, or association, is dependent, amongst others, on ionic binding, hydrogen bridges and hydrophobic effects (displacement of water). The strength of the binding between the binding pocket of an antibody and an epitope is designated as “affinity”.

Antibodies which are capable of agglutinating red blood cells under all conditions are designated agglutinins or complete antibodies (in general IgM antibodies). Antibodies which bind to (sensitize) erythrocytes, but cause no direct agglutination, are called incomplete antibodies (in general IgG antibodies).

Red blood cell antigens and their corresponding antibodies are often detected by means of agglutination reactions, which can take place in a physiological salt solution. In practice, agglutination tests can be rendered more sensitive by using, for example, a medium having a low ionic strength, proteolytic enzymes (e.g. bromelin, papain or ficin), polycations (e.g. polybrene), macromolecules (e.g. albumin), or polymers (e.g. polyethylene glycol (PEG) or dextrane). A large number of serological tests is known.

Common, generally used serological tests are the tube method, micro column tests, and tests in micro plates. These techniques can be further divided into techniques based on agglutination, i.e. complex formation, and techniques based on a solid-phase (affinity) principle.

Furthermore, blood typing tests based on DNA techniques are available, for example, blood group genotyping, their application in the field of serology is gaining more importance. In addition, techniques based on fluorescent labels or magnetic beads are available.

When considering the present major clinical applications in the field of serology, the tube method, micro column tests, and tests in micro plates tests are widely used and will be further detailed below.

The tube method is a widely used test allowing prolonged incubations with antibodies. Erythrocytes, after a reaction with antibodies, can be sedimented, or centrifuged, to accelerate the agglutination reaction.

An important, and widely applied, variant of the tube method test is the antiglobulin test or Coombs test, described by Moreschi in 1908 and reintroduced in 1945 by Coombs et al. The Coombs test is based on the principle that erythrocytes loaded with, for example, incomplete antibodies of the IgG type can be agglutinated through the addition of antiglobulin serum. In the test, three phases can be distinguished.

The first phase is the sensitization phase. During this phase, antibodies bind to, or associate with, the corresponding antigen structures on the red blood cells (sensitization of red blood cells). When binding, thereby forming carrier (erythrocyte)-bound analytes (antibodies), has occurred, a second phase is initiated, also designated as the wash phase. In this wash phase, substantially all non-bound or non-associated antibodies are removed from the incubation mixture.

The third phase is designated as the antiglobulin phase, in which antiglobulin serum is added to the washed sensitized, i.e., antibody loaded, cells. This causes binding of sensitized cells to each other resulting in the formation of complexes or agglutinates comprised of clustered, i.e., from about 50 to thousands, erythrocytes.

When performing the Coombs test, it is necessary, before adding the antiglobulin serum, to wash very thoroughly and frequently, and thus this step is very time consuming Insufficient removal of non-bound antibodies can lead to inactivation of the antiglobulin serum. Other disadvantages of the test are the need for promptly reading the results by a trained professional, that the test results cannot be preserved, the test is less reproducible because of manual reading of the test results, and difficulties in automating the test.

As indicated, the washing step in the Coombs test is very time consuming. An improvement was achieved by LaPierre et al. and European Patents 0 194 212 and 0 305 337 using a micro column of inert and solid particles for retaining agglutinates upon centrifugation, while the serum remains on top of the micro column and non-agglutinated cells can readily pass through the micro column.

In this test system, use is made of small columns filled with Sephadex® gel. Use can be made of columns comprising antibodies (e.g. for blood group typing) or no antibodies (e.g. for reverse ABO typing).

In order to perform a Coombs test, use is made of gel columns containing antiglobulin serum. After incubation, the gel columns are centrifuged. In case of a negative reaction, i.e., no carrier-bound analyte complexes are formed, all (individual) erythrocytes will end up at the bottom of the micro column; if the test is positive, the erythrocyte complexes, or agglutinates, will be more or less retained by the column, i.e., will, after centrifugation, be visible on top or somewhere in the column. In case of weak reactions, erythrocytes will sediment partly resulting in erythrocytes in the column and at the bottom of the column. This test is marketed by BioRad (ID Microtyping System) and Grifols (DianaGel, DG Gel® cards)

A comparable micro column system has been described using non-compressible micro particles instead of gel material as inert material to retain the complexes or agglutinates formed (European Patents 0 485 228, 0 725 276, 0 755 719 and U.S. Pat. Nos. 5,552,064 and 5,650,068). This test is marketed by Ortho (Biovue System), using glass beads as non-compressible micro particles.

Another micro column system has been described using an alternative agglutination reagent, including synthetic particles (U.S. Pat. No. 6,203,706). Such a test is marketed by BioRad (ID-PaGIA test).

A number of drawbacks are associated with the above micro column systems. For example, a special centrifuge is required for correct performance of the test. Also, special reading equipment is required for automatic reading of the test as well as special equipment for automation of the entire test.

A general disadvantage of the above micro column agglutination tests is the occurrence of a smear of erythrocytes or synthetic particles, especially when weak reactions are tested, resulting in uncertainty when reading and interpreting the strength of the reaction results, both at reading by the naked eye and at automated reading of the results.

Another general drawback of the above micro column agglutination tests is that shear forces during centrifugation can cause weak agglutinates to disintegrate into several small erythrocyte clusters or rosettes (or, in extreme cases, into individual (sensitized) erythrocytes), that will not be detected since they are too small to be sieved by the gel particles and therefore will sediment at the bottom, resulting in an increase of the number of false negative reactions.

Another approach in (erythrocyte and thrombocyte) serology testing is based on the solid-phase (affinity) principle as an alternative for direct (complex formation or agglutination without a bridging reagent) and indirect (complex formation or agglutination with a bridging reagent) reactions for blood group typing, antibody screening, antibody identification and cross match. Applications and advantages of the use of affinity solid-phase techniques in serology have been described by Rosenfield, 1976 and U.S. Pat. No. 4,275,053. Here, amongst others, red blood cells were used which had been coupled to the surface of plastic tubes.

Other systems have been described by Plapp et al., 1984, Bayer et al. (U.S. Pat. No. 4,608,246), Rachel et al., 1985, Plapp et al., 1986 and Uthemann et al. (U.S. Pat. No. 4,925,786, and European Patent 0 363 510).

Microplates in combination with the solid-phase principle are used by, for instance, BioRad (Erytype for typing of red cell antigens and Solidscreen II for antibody diagnostics), Immucor Inc. (Capture-R system for antibody screening and identification, Capture-P system for thrombocyte antibody detection and cross match) and Sanquin Reagents B.V. (Maspat system for thrombocyte cross match). Another application is the MAIPA (Monoclonal Antibody Immobilization of Platelet Antigens) test for the detection of thrombocyte antibodies. Microplates can also be used in a test format with magnetic beads being coupled to red cells. In this way, no centrifuge step is required, since red cells (carrying antibodies or not) are simply separated from the reaction mixture by applying a magnetic field followed by either binding to the bottom surface of the well or collection in the center of the well (QWALYS, Diagast).

The solid-phase affinity tests are not limited to microplates. Affinity gel tests for blood group typing and antibody detection have been described by Pernell (Sanofi Pasteur/BioRad, European Patent 0 594 506), Gamma/Immucor (WO 95/31731 and WO 98/16831 and U.S. Pat. Nos. 5,665,558 and 5,905,028) and Van der Donk et al. (Sanquin, European Patent 1 064 556).

A major disadvantage of the above described affinity gel test systems is that a relatively high amount of costly ligand molecules is necessary, while only a fraction thereof, present on the outer surface of the gel particles, is effectively utilized for interaction, or association, with erythrocytes.

Another disadvantage is that a-specific interactions can occur between (non sensitized) red blood cells and the gel matrix and/or the immobilized ligands, resulting in apparent binding of red blood cells to the gel matrix and thereby in an increase in the amount of false positive reactions.

Many serological tests on erythrocytes use the red color of these erythrocytes for visualizing the reaction result. This, however, is not possible for serological tests on thrombocytes. A possible approach here (applicable for erythrocytes as well) is the use of fluorescent labels. Cells carrying typing antibodies (or not) against a particular antigen are incubated with a secondary compound, carrying a fluorescent dye and capable of reacting specifically with the typing antibody bound to the cell. Next, antigen positive cells carrying a typing antibody can be identified using flow cytometry analysis. This technique can for instance be applied to thrombocytes in the Platelet Immunofluorescence Test (PIFT) described by Von dem Borne et al., 1978

In a similar way, direct or indirect immunofluorescence staining followed by flow cytometry analysis can be applied to erythrocytes for blood group typing, using single tubes or micro plate wells for each separate typing reaction.

A major disadvantage of all tube, micro column and micro plate tests described above, is that the amount of samples to be tested per analysis run is limited by the capacity of the tube, micro column or microplate and/or the centrifuge to be used for spinning the tubes, micro columns or micro plates and/or the reader or flow cytometer for visualizing the reaction result. Every sample in these tests requires a separate tube, micro column or micro plate well.

Another disadvantage of these tests is that relatively large amounts of sample material are required for performing the tests, thereby limiting the amount of tests to be carried out in case of particular clinical and/or neonatal samples.

Array methods, allowing simultaneous analysis of a larger number of samples by spotting reactive components on a chip format, have been described for DNA genotyping and are used, for example by Beckmann Coulter (GenomeLab SNPstream) and BioArray Solutions (BeadChip). DNA genotyping is limited to typing of blood groups, however, screening and identification of antibodies cannot be performed.

An array method for the detection of thrombocyte antibodies has been described, using a micro-bead assay on the Luminex platform, for example PAKTM-Lx (GTI Diagnostics® and Gen-Probe).

Another array method for the detection of red cell antibodies, as well as for blood group typing has been described, using spotted (fragments of) red blood cells in the (announced) Mosaiq system (Quotient Biosciences).

A major disadvantage of these array methods is that dedicated equipment and disposables are required for performing multiplex assays.

Considering the above, it is an object of the present invention, amongst others, to obviate at least part of the above disadvantages associated with the known (serology) test systems.

### SUMMARY OF THE INVENTION

According to the present invention, this object is met as outlined in the appended claims.

Especially, this object is met by the present invention by providing a single tube, i.e. a single container, aqueous preparation, such as a buffer, isotonic solution or physiological salt solution, comprising a panel of differently labeled serologically relevant blood cell types for blood serology, wherein the differential labeling comprises a label selected from at least two different labels and association of the selected label with the serologically relevant blood cell types in different amounts.

Formulated differently, the present invention provides a single aqueous composition, such as a buffer or isotonic salt solution, of blood cells forming a comprehensive panel based on the blood group serology to be determined or tested. Generally, 100 to 500,000, such as at least 500 for example 1,000 or 1,500 to 250,000 blood cells of each blood cell type are sufficient for blood serology.

The present inventors have surprisingly found that using a limited set of labels, such as 2, 3 or 4, in combination with associating different amounts of label to the blood cells allows for discrimination of 6 to 216 blood cell types, such as 8, 10, 12, 14, 16, 18, 20, 30, 40, 50, 60, 80, 100 or more blood cell types, in a single assay or test.

Preferably, the serology using the present panel of differently labeled serologically relevant blood cell types is used in combination with flow cytometry.

According to the present invention, the blood cells forming the present panel are preferably erythrocytes, thrombocytes and/or white blood cells, more preferably erythrocytes or thrombocytes, most preferably erythrocytes.

The present labels are preferably fluorescent labels but also color, enzymatic or radioactive labels are contemplated within the context of the present invention.

The present single tube preparation can be readily prepared using methods comprising the steps of:
individually incubating serologically relevant blood cell types with a label selected from at least two labels at different concentrations of the selected label under conditions allowing association of the label to the serologically relevant blood cells types;
removing non-associated label;
combining the individually incubated serologically relevant blood cell types in a single tube preparation.

Preferably, in the above incubation, the concentrations of the label are in the range of 0 to 100 mg/ml. Within this range, the different concentrations result in discernable amounts of label associated with the blood cell types. For example, individually incubating the blood cell types with concentrations of 0, 20, 40 and 60 mg/ml with a single label results in 4 discernable blood cell types in the present single tube preparation.

In the art, numerous methods are known to associate, attach or bind labels to cells such as direct labeling using fluorescein isothiocyanate or indirect labeling using biotin/streptavidin.

The present panel of differently labeled serologically relevant blood cell types allow for serology or blood typing methods comprising the steps of:
providing the present single tube preparations;
incubating a serology relevant antibody sample, whole blood, plasma, a serum or serum derived sample of an individual in need of blood serology with the single tube preparation under conditions allowing association of the serology relevant antibody sample, the serum or serum derived sample with the differently labeled serologically relevant blood cells types;
removing non-associated serology relevant antibody sample, the serum or serum derived sample;
analyzing the association of the panel of differently labelled serologically relevant blood cells types with the serology relevant antibody sample, the serum or serum derived sample.

Examples of serology relevant antibody samples for testing the presence or absence of human blood cell antigens and/or complement components are samples containing monoclonal and/or polyclonal antibodies or fragments thereof, with a corresponding specificity, originating from human or animal sources. Generally, the serum or serum derived sample allows for testing the presence or absence of human blood cell antibodies, including the isotype of antibodies present.

The present analyzing preferably comprises flow cytometry allowing automated high-throughput data collection and subsequent computerized data analysis and presentation.

According to another preferred embodiment, analyzing the differentially labeled panel cells that reacted with the sample comprises detection using sensors in a microfluidics system, allowing analysis with lower amounts of reagents, automated data collection and subsequent computerized data analysis and presentation.

According to another preferred embodiment, analyzing the differentially labeled panel cells that reacted with the sample comprises immobilizing sample serology relevant cells on a solid support, capturing an image and subsequent computerized data analysis and presentation.

Further automation and high-throughput are preferably provided by performing the present methods in multi-well or microtiter plates.

In the present method, after removing but before analyzing further labels are added, preferably labelled antibodies capable of detecting human antibodies and/or complement components. Examples of suitable antibodies in this preferred step of the present method are anti-human immunoglobulin antibodies or anti-human complement antibodies or fragments thereof.

The present serology method is preferably used for serologically characterizing an individual in need of a blood transfusion, serologically characterizing a blood donor or preventive serologically characterizing an individual. For example, screening and identification of red cell and/or platelet antibodies present in the serum of donors and patients, selecting suitable donors for a transfusion by cross-matching between possible donors and recipient, screening and identification of antibodies developed during pregnancy.

### DETAILED DESCRIPTION OF THE INVENTION

In blood group serology the serum of a recipient of a blood transfusion must be screened for the presence of red cell antibodies. If an antibody is detected, it must be identified in order to be able to select donor blood which is negative for the corresponding blood group.

For screening of the serum for the presence of red cell antibodies a panel consisting of 2 or 3 different red cell suspensions is required, carrying as a whole all clinical relevant blood groups and preferably in homozygous expression. If no red cell antibodies are present in the serum, reactions with the 2 or 3 different screening panel red cell suspensions will be negative and no additional measures are necessary for the selection of compatible donor blood.

However, if a red cell antibody is present in the serum, a positive reaction with one or more screening panel red cells suspensions is observed and the antigenic specificity must be established. For this subsequent identification of red cell antibodies found during screening, an identification panel of at least 8 up to about 15-20 different red cell suspensions is required, carrying all clinical relevant blood groups in various combinations and including red cells negative for particular blood groups. Based on the reaction pattern of the recipient serum with all individual red cell suspensions, the identity of the red cell antibody or antibodies can then be established.

Although the 2 or 3 different red cell suspensions of a screening panel are visually indistinguishable they could theoretically be mixed together in order to save some sample material (recipient serum), reagent red cells, disposables and analysis time, since one is only interested in a possible positive reaction with any of the 2 or 3 different red cell suspensions. However, by mixing these 2 or 3 different red cell suspensions the blood group antigens present are diluted, since not every blood group is present on all 2 or 3 cells, which may lead to false negative reactions. Since the 8 to 15-20 different red cells of the identification panel are visually indistinguishable too, these cannot be mixed together since one must know the outcome of the reaction of the recipient serum with every single panel red cell suspension in order to be able to establish the identity of the red cell antibody or antibodies, thereby requiring relatively much sample material, reagent red cells, disposables and analysis time.

The present inventors have surprisingly found a method to label at least 6 such as 8 to 15-20 different red cells of an identification panel individually and uniquely, allowing mixing them all together in a single vessel, and still distinguish their reactivity with recipient serum with high sensitivity, thereby saving (patient) sample material, reagent red cells, disposables and analysis time.

In order to be able to distinguish up to 15-20 different red cells one would have to label these cells with 15-20 different markers like coloring agents, radioactive tracers, enzymes or fluorescent dyes. Selection of these high numbers of for example different fluorescent dyes is not very practical in terms of measuring them simultaneously, since specialized equipment/reagents at high costs would be required. The present inventors have surprisingly found that it is possible to use only 2 different fluorescent dyes to (double) label up to 25-36 different red cells distinguishably while allowing simultaneous identification, by using 5 to 6 different concentrations per fluorescent dye. In this way, a single tube matrix/array for red cell antibody identification is generated.

In a reaction vessel, incubation of a mixture of different, distinguishable carriers, such as fluorescently (single, double or non-)labeled erythrocytes, and analytes, such as antibodies, results in the formation of carrier-bound analytes, such as antibody sensitized erythrocytes, for one or more of the different carriers present in the mixture.

Next, carriers, preferably red blood cells, sensitized with analytes, preferably complement factors and/or IgG antibodies and/or IgM antibodies and/or IgA antibodies, are allowed to react with secondary analytes, preferably anti-complement antibodies and/or anti-IgG antibodies and/or anti-IgM antibodies and/or anti-IgA antibodies, loaded with a (distinguishable) fluorescent dye in order to form carrier-bound secondary analytes.

It is of importance to note that substantially no complex formation, i.e., agglutination of carrier-bound analytes, occurs in the reaction vessel upon incubation with analytes. However, this inherently implies that, in the case of IgM antibodies, the spontaneous formation of complexes or agglutinates in the reaction vessel must be substantially prevented. The skilled person, by following the Heidelberger curve, is readily capable to choose the concentrations of carrier and analyte in such a way that complex formation or agglutination is substantially prevented, for example, by lowering of the concentration of red cell antibodies by dilution.

The formation of complexes or agglutinates by the secondary analytes in the reaction vessel must also be substantially prevented, for example, by lowering of the concentration of secondary analytes by dilution or by using fragments of secondary analytes.

After the incubation phases in the reaction vessel, unbound (secondary) analytes are removed from the incubation mixture, for example by centrifugation and aspiration or filtration.

Next, the mixture of different carriers with bound (secondary) analytes (or not) is analyzed by flow cytometry analysis, allowing to determine in a single run which of the different carriers present have bound (secondary) analytes and which have not. By including at least 8 up to 15 different carriers in the mixture, the antigenic specificity of the analyte(s) can be determined in a single assay run, saving sample material, reagent red cells, disposables and analysis time.

Using the method according to the present invention, it was found possible to simultaneously demonstrate the presence of IgG type antibodies and/or IgM type antibodies and identify their antigenic specificity with a high sensitivity.

Instead of mixing multiple different carriers with a known antigen pattern and incubating this mixture with unknown analytes, the method can also be applied the other way around, that is mixing multiple different carriers with an unknown antigen pattern and incubating this mixture with known analytes.

In this way, it was found possible to simultaneously demonstrate the presence or absence of different antigens on every individual carrier, for example in simultaneously detecting the presence or absence of certain blood group antigens in multiple donors.

Furthermore, the amount of red cells needed for antibody identification or blood group typing was decreased considerably (50-100 fold), as compared with current serological test procedures based on agglutination.

The present method as defined above provides many advantages over the serology test systems of the prior art, especially with respect to an increased sensitivity, a reduced requirement of sample material, reagents, disposables and analysis time and a high automation potential of the test.

Considering the above advantageous properties of the method, a method is described for simultaneous detection of carrier-bound analytes comprising:
a) labelling of multiple carriers with different fluorescent dyes in various concentrations;
b) mixing of the labelled carriers;
c) loading mixed carriers and analyte into a vessel;
d) incubating said mixed carriers and said analyte in said vessel for forming (mixed) carrier-bound analytes;
e) removing, by centrifugation and aspiration or filtration, non-bound analytes;
f) loading secondary analytes carrying a (distinguishable) fluorescent dye in said vessel;
g) incubating said (mixed) carrier-bound analytes and said secondary analytes for forming (mixed) carrier-bound secondary analytes;
h) removing, by centrifugation and aspiration or filtration, non-bound secondary analytes;
i) detecting the presence of carrier-bound secondary analytes within the mixture of labelled carriers by flow cytometry analysis
j) identification of bound analytes by (software) interpretation

In the reaction method, the incubations can be performed in microplates, which can be closed at the bottom or have a filter based bottom plate using labels such as CELLTRACE CFSE, CELLTRACE violet, fluorescently tagged N-Hydroxysuccinimide esters (for example FITC, Pacific Blue) using concentrations varying from 1 to 60 mg/ml. The secondary analyte can be loaded with PE or APC as a fluorescent label and the incubation of mixed carriers and analytes can be carried out during 5 minutes to 30 minutes at 2 to 37° C.

The incubation of (mixed) carrier-bound analytes and secondary analytes according to the present method preferably comprises 5 to 30 minutes at 2 to 37° C.

In another preferred embodiment, the carriers, preferably red blood cells, sensitized with analytes, preferably complement factors and/or IgG antibodies and/or IgM antibodies and/or IgA antibodies, are allowed to react (or not) with secondary analytes, preferably anti-complement antibodies and/or anti-IgG antibodies and/or anti-IgM antibodies and/or anti-IgA antibodies attached to a solid support, preferably a coated surface, an array of coated surfaces, or magnetic beads, in order to capture carriers sensitized with analytes.

In one preferred embodiment, sensors, preferably microscopic imaging followed by digital image analysis, determine the presence (or absence) of carriers-sensitized with analytes, allowing to determine in a single run which of the different carriers present have bound (secondary) analytes and which have not. By including at least 8 up to 15 different carriers in the mixture, the antigenic specificity of the analyte(s) can be determined in a single assay run, saving sample material, reagent red cells, disposables and analysis time.

The present invention will further be detailed in the following examples comprising, and describing, preferred embodiments of the present invention. In the examples, reference is made to the appended figures, wherein:

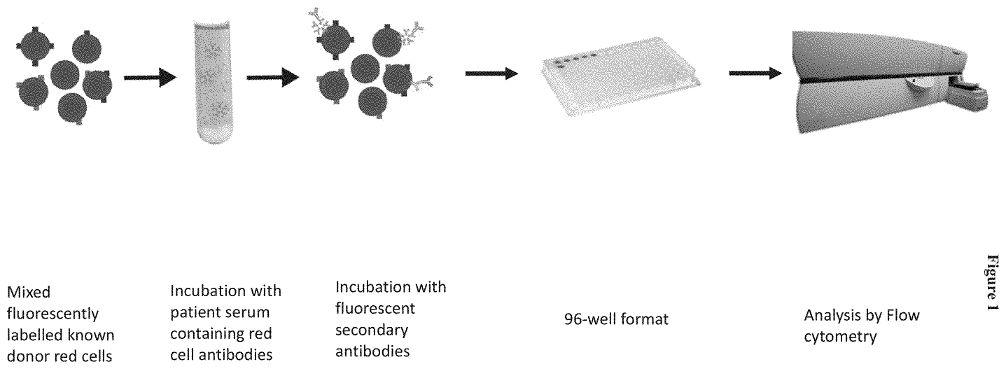

FIG. 1: shows a schedule of the a method for simultaneous detection of carrier-bound analytes, in this case erythrocytes with bound antibodies. Red blood cells from different known donors are (double) labelled (or not) and mixed together. Next, the mixed fluorescently labelled red cells are incubated with a sample containing analytes (for example IgG type or IgM type antibodies) and consecutively incubated with fluorescently labelled secondary antibodies (for example anti-IgG antibodies or anti-IgM antibodies or fragments thereof). After that, red cells with bound (secondary) antibodies are analysed by flow cytometry.

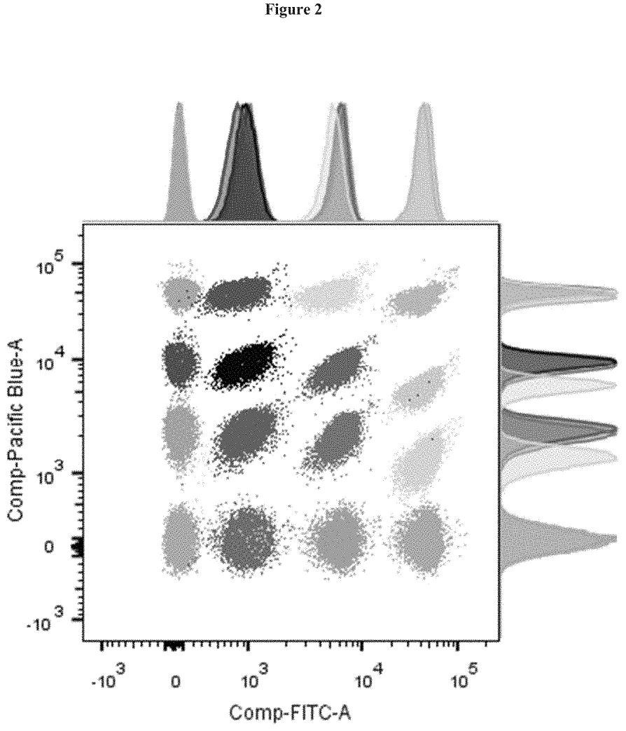

FIG. 2: shows how fluorescent dyes can be used to differentially label erythrocytes to generate a 4×4 matrix, which does not interfere with antigen detection and is stable over 9 weeks of storage. Red blood cells from 16 known donors were labelled using different concentrations of Fluorescein isothiocyanate isomer I (FITC) and Pacific Blue Succinimidyl Ester to generate a matrix as assessed by flow cytometry. The 16 differentially labelled donor red cells can be easily discriminated.

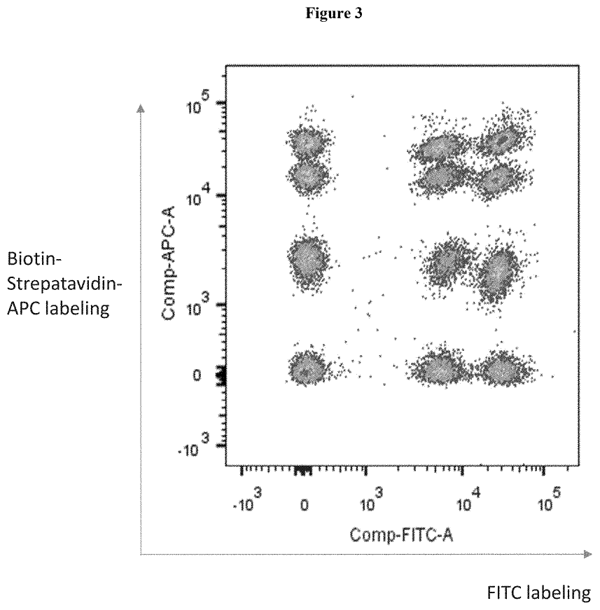

FIG. 3: shows a 2D plot of flow cytometry analysis of mixed red blood cells from 12 known donors, single, double or non-labelled fluorescently using different concentrations of FITC and Biotin/Streptavidin-APC. The 12 differentially labelled donor red cells can be easily discriminated in a 4×3 matrix.

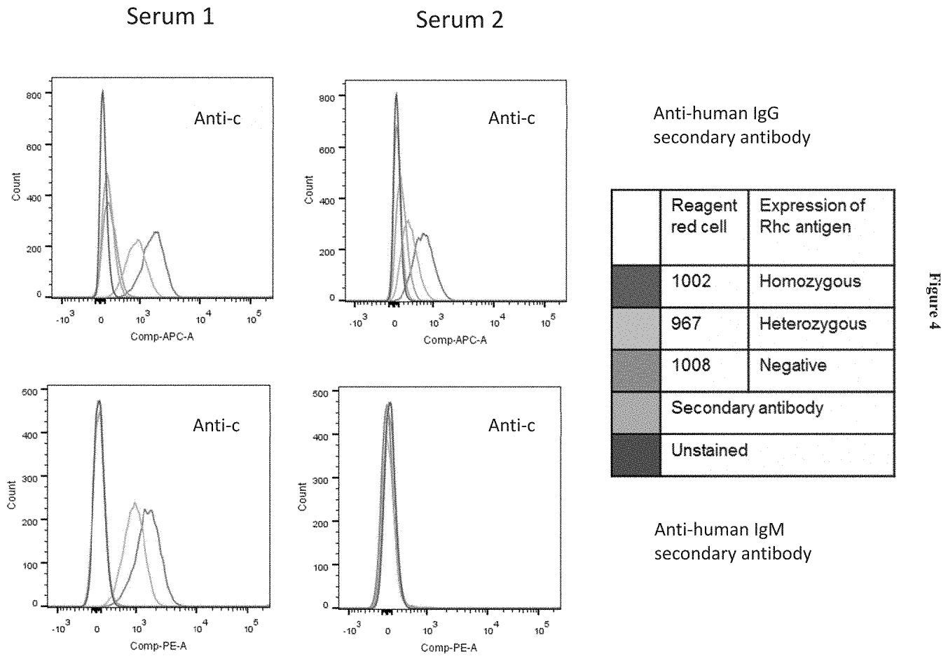

FIG. 4: shows detection of an anti-Rhesus c antibody in two known patient serum samples. Histograms display the detection of IgG and/or IgM antibodies against Rhc within these serum samples. Colors represent three different reagent red cells (1002, 967, 1008) or controls as indicated in the inlet. Serum 1 contains IgG type and IgM type antibodies against Rhc, while serum 2 contains only IgG type antibodies against Rhc. Reagent red cells with a heterozygous expression of the Rhc antigen display a lower signal than reagent red cells with a homozygous expression.

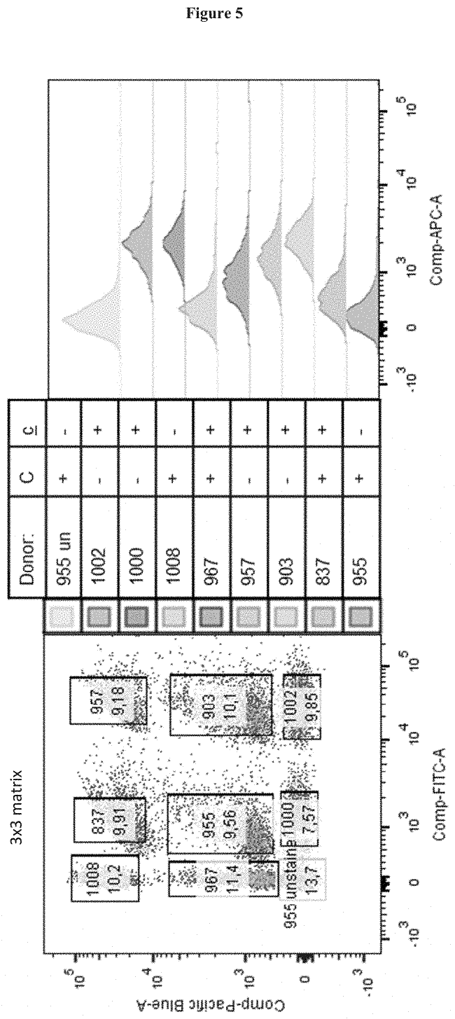

FIG. 5: shows the results of flow cytometry analysis for identification of a red cell antibody. A known patient serum sample, containing an anti-Rh c antibody, was incubated with mixed red blood cells from 8 known donors, single, double or non-labelled fluorescently using different concentrations of FITC and

Biotin/Streptavidin-Pacific Blue. Secondary antibody labeled with Biotin/Streptavidin-APC was used to detect anti-Rhc antibody associated with red blood cells in the mixture. Red cells positive for the Rh c antigen can be easily discriminated in the 3×3 matrix: red cell numbers 1002, 1000, 957 and 903 have a homozygous expression of the Rh c antigen and display the highest signal; red cell numbers 967 and 837 have a heterozygous expression of the Rh c antigen and display a lower signal; red cell numbers 1008 and 955 are negative for the Rh c antigen.

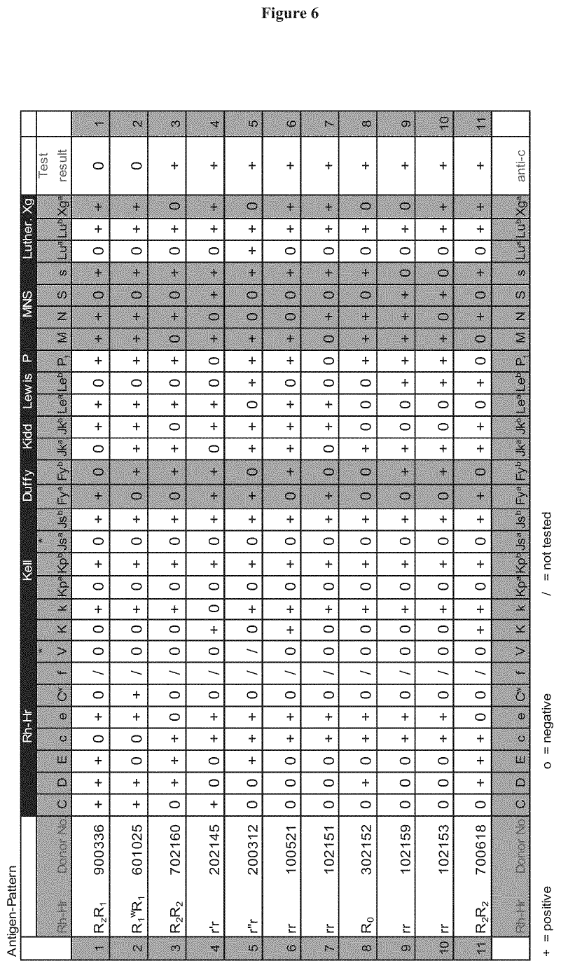

FIG. 6: shows an antigen pattern of an identification panel of 11 different reagent red cells, indicating the (homozygous or heterozygous) presence or absence of clinically relevant blood group antigens, and the test results of the reaction with a known patient serum sample containing an anti-Rhc antibody, according to the present method. From the reaction pattern displayed in the second last column the presence of an anti-Rhc antibody can be confirmed.

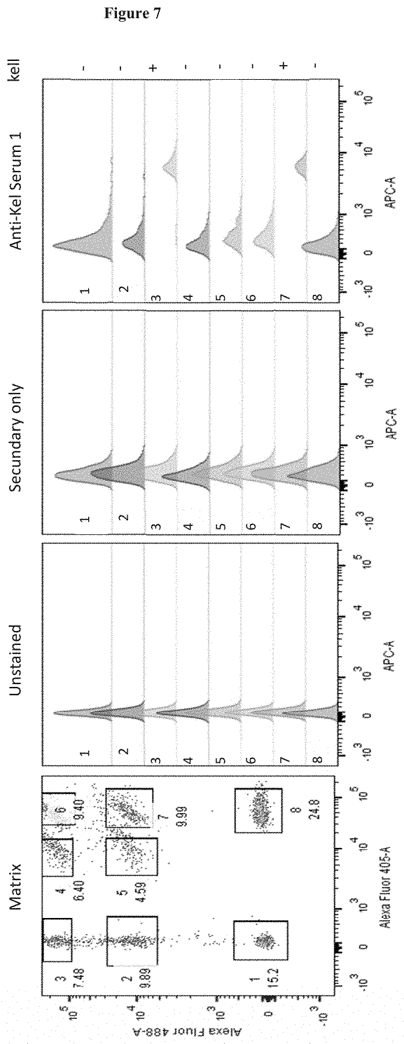

FIG. 7: shows the results of flow cytometry analysis for identification of an anti-K(ell) red cell antibody. A known patient serum sample, containing an anti-K antibody, was incubated with mixed red blood cells from 8 known donors, single, double or non-labelled fluorescently using different concentrations of Alexa Fluor 405 and Alexa Fluor 488. Secondary antibody labeled with Biotin/Streptavidin-APC was used to detect anti-K antibody associated with red blood cells in the mixture. Red cells number 3 and 7 are positive for the K antigen and can be easily discriminated in the matrix from the other cells that are negative for the K antigen.

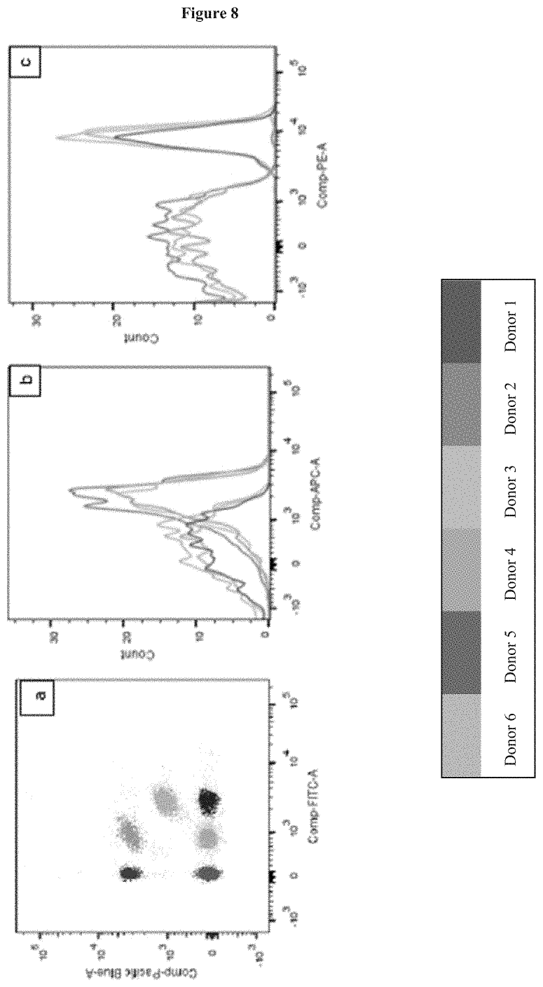

FIG. 8: shows the results of flow cytometry analysis for blood group typing of the Fya and Fyb antigen on a mixed red cell population of 6 donors by the present method. The differently labelled red cells from 6 donors can be easily discriminated and the presence or absence of the Fya and Fyb antigen could clearly be demonstrated for each individual donor: donor 1, 2 and 3 are positive for the Fya antigen and negative for the Fyb antigen; donor 4, 5 and 6 are negative for the Fya antigen and positive for the Fyb antigen.

### EXAMPLES

### Example 1

Red blood cells from 16 different known donors were washed and suspended in phosphate buffered saline (PBS) with 2% bovine serum albumin (BSA, Sigma-Aldrich). For fluorescent labeling of red blood cells stock solutions of 1 mg/ml were prepared for biotin and FITC. These 16 different red blood cell populations (about 50000 cells per donor) were incubated with various concentrations (0, 6, 20 and 60 mg/ml) of biotin and FITC for 30 minutes at 37° C. while shaking at 350 rpm. After incubation red cells were washed with PBS/2% BSA.

Next, red blood cells were incubated for 30 minutes in the dark at 4° C. with streptavidin-Pacific Blue, binding to biotin. After washing, red blood cells were analyzed by flow cytometry on 5 or 3 Laser flow cytometers with High Throughput Sampler (5L Fortessa+HTS, 3L Canto II+HTS and 5L LSR II+HTS). Obtained data were analyzed using Flowjo software. According to FIG. 2, in this way a matrix of 16 single, double or non-labelled red blood cells can be created, wherein the differentially labelled erythrocytes can be easily discriminated.

### Example 2

Red blood cells from 12 different known donors were labelled as described in Example 1, now using 0, 20, and 60 mg/ml FITC and 0, 6, 20, and 60 mg/ml biotin and streptavidin-APC instead of streptavidin-Pacific Blue. After washing, the 12 different red blood cell populations were mixed and analyzed by flow cytometry as described in Example 1. According to FIG. 3, in this way a matrix of 12 single, double or non-labelled red blood cells can be created, wherein the differentially labelled erythrocytes can be easily discriminated.

### Example 3

Red blood cells from 3 different known donors (homozygous, heterozygous and negative for the Rhc antigen, respectively) were washed and suspended in PBS/2% BSA. These 3 different red blood cell populations were incubated for 15 minutes at 37° C. with 2 known patient sera containing anti-Rhesus c antibodies. After incubation red cells were washed three times with PBS/2% BSA. Next, red blood cells were incubated for 30 minutes in the dark at 4° C. with fluorescent secondary antibodies (anti-human IgG, APC labelled and anti-human IgM, PE labelled).

After washing three times with PBS, red blood cells were analyzed by flow cytometry as described in Example 1. According to FIG. 4, histograms display the detection of IgG and/or IgM antibodies against Rhc within these two patient serum samples. Colors represent the three different donor red cells (1002, 967, 1008) or controls as indicated in the inlet. Serum 1 contains IgG type and IgM type antibodies against Rhc, while serum 2 contains only IgG type antibodies against Rhc. As expected, donor red cells with a heterozygous expression of the Rhc antigen display a lower fluorescence signal than donor red cells with a homozygous expression.

### Example 4

Red blood cells from 8 different known donors (homozygous, heterozygous and negative for the Rhc antigen, respectively) were labelled as described in Example 1, using different concentrations of FITC and Biotin/Streptavidin-Pacific Blue. After washing, the 8 differently labelled red blood cell populations and one unlabeled control were mixed and suspended in PBS/2% BSA. Next, these mixed red cells were incubated for 15 minutes at 37° C. with the patient serum containing anti-Rhc antibody. After incubation mixed red blood cells were washed three times with PBS/2% BSA.

Next, mixed red blood cells were incubated for 30 minutes in the dark at 4° C. with fluorescent secondary antibodies (anti-human IgG, APC labelled). After washing three times with PBS, mixed red blood cells were analyzed by flow cytometry as described in Example 1. According to FIG. 5, 6 red blood cell populations carrying the Rhc antigen and thereby reactive with the anti-Rhc antibody could easily be detected within the 3×3 matrix due to fluorescence of the APC label, 2 antigen negative red blood cell populations and the control showed no fluorescence of the APC label. Red blood cells with a heterozygous expression of the Rhc antigen display a lower fluorescence signal than red blood cells with a homozygous expression.

### Example 5

11 different reagent red blood cells were labelled as described in Example 2. Red cells from one patient, known for having an IgG anti-Rhc antibody in the serum, were not labelled and used as autocontrol. After washing, the 11 differently labelled reagent red blood cells and patient red blood cells were mixed and suspended in PBS/2% BSA. Next, these mixed red blood cells were incubated for 15 minutes at 37° C. with the patient serum containing anti-Rhc antibody. After incubation mixed red blood cells were washed three times with PBS/2% BSA.

Next, mixed red blood cells were incubated for 30 minutes in the dark at 4° C. with fluorescent secondary antibodies (anti-human IgG, APC labelled). After washing three times with PBS, mixed red blood cells were analyzed by flow cytometry as described in Example 1. 9 reagent red blood cells carrying the Rhc antigen and thereby reactive with the anti-Rhc antibody could easily be detected within the 4×3 matrix due to fluorescence of the APC label, 2 Rhc antigen negative reagent red blood cells and the autocontrol showed no fluorescence of the APC label. According to FIG. 6, comparison of the test results with the antigen profile of the 11 reagent red blood cells confirms the presence of an anti-Rhc antibody.

### Example 6

Red blood cells from 8 different known donors (homozygous, heterozygous and negative for the K(ell) antigen, respectively) were labelled as described in Example 1, now using different concentrations of Alexa Fluor 405 and Alexa Fluor 488. After washing, the 8 differently labelled red blood cell populations were mixed and suspended in PBS/2% BSA. Next, these mixed red blood cells were incubated for 15 minutes at 37° C. with the patient serum containing anti-K antibody. After incubation mixed red blood cells were washed three times with PBS/2% BSA.

Next, mixed red blood cells were incubated for 30 minutes in the dark at 4° C. with fluorescent secondary antibodies (anti-human IgG, APC labelled). After washing three times with PBS, mixed red blood cells were analyzed by flow cytometry as described in Example 1. According to FIG. 7, 2 red blood cell populations carrying the K antigen and thereby reactive with the anti-K antibody could easily be detected within the matrix due to fluorescence of the APC label, 6 K antigen negative red blood cell populations showed no fluorescence of the APC label.

### Example 7

7 more patient serum samples with (formerly) known (weak) red cell antibodies were analyzed as described in Example 5, now using anti-human IgG, APC labelled and anti-human IgM, PE labelled as fluorescent secondary antibodies, incubating for 5 min in the dark at 4° C. and using (7 wells of) a microplate to prepare all 7 patient samples. According to Table 1 all red cell antibodies could still be identified in these 7 patient serum samples, whereas the reference test (indirect anti-globulin test (tube method)) could only detect antibodies in 3 patient serum samples. Moreover, with the present method, the presence of IgG type and/or IgM type red cell antibodies could directly be established in the same assay run.

TABLE 1

| | | Reference test | | | | | |
|---|---|---|---|---|---|---|---|
| Patient sample | | (tube test (IAT)) | | Present method | | | |
| sample | antibodies | test | titer | test | signal | IgG type | IgM type |
| no. | present | result | strength | result | strength | antibodies | antibodies |
| 504698 | anti-E | Pos | 1:1 | pos | good | yes | no |
| 504814 | anti-D | Neg | — | pos | weak | yes | no |
| 504882 | anti-c | Pos | 1:8 | pos | good | yes | yes |
| 504936 | anti-E | Neg | — | pos | weak | yes | no |
| 505192 | anti-E | Pos | 1:4 | pos | good | yes | yes |
| 505194 | anti-c | Neg | — | pos | good | yes | no |
| 505217 | anti-Jka | Neg | — | pos | weak | yes | no |

### Example 8

Red blood cells from 6 different donors were labelled as described in Example 2, now using 0, 6, and 20 mg/ml FITC and 0, 6, and 20 mg/ml biotin and streptavidin-APC in different combinations. After washing, the 6 different red blood cell populations were mixed and suspended in PBS/2% BSA. Next, these mixed red blood cells were incubated for 15 minutes at 37° C. with IgG anti-Fya typing antibodies and IgM anti-Fyb typing antibodies. After incubation the mixed red blood cells were washed three times with PBS/2% BSA. Next, the mixed red blood cells were incubated for 30 minutes in the dark at 4° C. with fluorescent secondary antibodies (anti-human IgG, APC labelled and anti-human IgM, PE labelled). After washing three times with PBS, the mixed red blood cells were analyzed by flow cytometry as described in Example 1.

According to FIG. 8, histograms display the presence or absence of Fya and Fyb antigen on red blood cells of all 6 individual donors present in the mixture: donor 1, 2 and 3 are positive for the Fya antigen and negative for the Fyb antigen; donor 4, 5 and 6 are negative for the Fya antigen and positive for the Fyb antigen. These typing results were confirmed by reference testing in the tube method. In a comparable way the presence of for example blood group antigens A, B, D, C, c, E, e, Cw, K, k, Jka, Jkb, M, N, S, s, Lua, Lub, Kpa, and Kpb could still be demonstrated after fluorescent labelling of red blood cells with FITC and biotin/streptavidin-APC.

### CONCLUSIONS

From FIG. 2 and FIG. 3, it is clear that a matrix of multiple red blood cell populations can be generated that are distinguishably fluorescently labelled, according to the present invention. For this, several fluorescent labels can be used in different concentrations, not interfering with blood group antigen detection and stable over time.

From FIGS. 4-7 and Examples 3-7 it is clear that, according to the present invention, red cell antibodies against blood group antigens present in a patient serum sample can be identified in a single tube test and with a higher sensitivity than with the Indirect Agglutination Test (tube method). Moreover, from FIG. 4 and Table 1 it is clear that, according to the present invention, in the same single tube test, also the antibody isotype (for example IgG or IgM) can be established.

From FIG. 1 and Example 7 it is clear that, according to the present invention, the presence of red cell antibodies in multiple patient sera can be identified in a high throughput manner, by using a single microplate well per patient sample. Moreover, from Example 1 and Example 7 it is clear that identification of red cell antibodies can be performed using a considerably reduced number of known donor red cells and a much lower amount of patient serum as compared to agglutination based assays, which is highly advantageous in situations where there is only a limited amount of test sample available.

From FIG. 8 and Example 8 it is clear that, according to the present invention, in a mixture of red blood cells from multiple donors the presence of different blood group antigens can be identified for each individual donor in a single tube test.

1. A single tube preparation comprising:
a panel of differently labeled serologically relevant blood cell types for blood serology,
wherein the differential labeling comprises a label selected from at least two different labels and association of the selected label with the serologically relevant blood cell types in different amounts.

2. The single tube preparation according to claim 1, wherein the blood serology comprises flow cytometry.

3. The single tube preparation according to claim 1, wherein the panel of differently labeled serologically relevant blood cell types comprises 6 to 216 differently labeled serologically relevant blood cell types.

4. The single tube preparation according to claim 1, wherein the differently labeled serologically relevant blood cells types are erythrocytes, thrombocytes and/or white blood cells.

5. The single tube preparation according to claim 1, wherein the at least two different labels are fluorescent labels, optical density detectable labels, or colometric labels.

6. The single tube preparation according to claim 1, wherein the at least two different labels are two or three different labels.

7. A method for providing a single tube preparation according to claim 1, the method comprising:
individually incubating serologically relevant blood cell types with a label selected from at least two labels at different concentrations of the selected label under conditions allowing association of the label to the serologically relevant blood cells types;
removing non-associated label; and
combining the individually incubated serologically relevant blood cell types in a single tube preparation.

8. The method according to claim 7, wherein the different concentrations are in the range of 0 to 100 mg/ml selected label.

9. The method according to claim 7, wherein the label is directly associated with the blood cells or indirectly through a bridging compound.

10. A single tube preparation obtainable by a method according to claim 7.

11. A method for blood serology, the method comprises the steps of:
providing the single tube preparation comprising a panel of differently labeled serologically relevant blood cell types according to claim 1;
incubating an serology relevant antibody sample, whole blood, plasma, a serum or serum derived sample of an individual in need of blood serology with the single tube preparation under conditions allowing association of the serology relevant antibody sample, the serum or serum derived sample with the differently labeled serologically relevant blood cells types;
removing non-associated serology relevant antibody sample, the serum or serum derived sample; and
analyzing the association of the panel of differently labelled serologically relevant blood cells types with the serology relevant antibody sample, the serum or serum derived sample.

12. The method according to claim 11, wherein analyzing comprises flow cytometry, microscopic optical detection, image capturing, or ImageStream.

13. The method according to claim 11, wherein incubating comprises incubation in a multi-well or microtiter plate.

14. The method according to claim 11, wherein after removing but before analyzing further labels labelled antibodies or fragments thereof are added, wherein said labelled antibodies or fragments thereof are capable of detecting human antibodies and or complement components.

15. The method according to claim 11, wherein blood serology comprises serologically characterizing an individual in need of a blood transfusion, serologically characterizing a blood donor or preventive serologically characterizing an individual.
